(19) United States
(12) Patent Application Publication
(10) Pub. No.: US 2007/0287752 A1
(43) Pub. Date: Dec. 13, 2007

(54) Ophthalmic Compositions Comprising A Branched, Glycerol Compound

(76) Inventors: Susan E. Burke, Batavia, NY (US); Joseph C. Salamone, Boca Raton, FL (US)

Correspondence Address: Joseph Barrera;Bausch & Lomb Incorporated, One Bausch & Lomb Place, Rochester, NY, 14604, US

(21) Appl. No.: 11/691,555

(22) Filed: Mar. 27, 2007

Related U.S. Application Data
(60) Provisional application No. 60/811,929, filed on Jun. 8, 2006.

(51) Int. Cl. A61K 31/16; A61K 31/08; A61K 31/135

(52) U.S. Cl. 514/625; 514/635; 514/667; 514/706; 514/722; 514/738

(57) ABSTRACT

An aqueous ophthalmic composition comprising a branched glycerol compound selected from the group consisting of a branched, glycerol monoalkyl ether, a branched, glycerol monoalkyl amine, a branched, glycerol monoalkyl sulfide, or any mixture thereof, present in a total amount of from 0.05 ppm to 1,000 ppm; a cationic antimicrobial component, and having an osmolality in a range from 200 mOsmol/kg to 400 mOsmol/kg. The presence of the branched glycerol compound enhances the biocidal efficacy of the aqueous ophthalmic composition containing one or more cationic antimicrobial components.

This application claims priority to U.S. provisional application Ser. No. 60/811,929 filed Jun. 8, 2006, the entire disclosure of which is incorporated herein by reference.

The invention relates to a contact lens composition comprising a branched, glycerol monoalkyl compound, and a cationic antimicrobial component, and the use of the composition to disinfect, clean, package or wet a contact lens.

### BACKGROUND OF THE INVENTION

Various antimicrobial agents are known for use as disinfectants or preservatives in ophthalmic compositions such as contact lens care solutions or eye drops, particularly eye drops formulated for use with contact lenses. The antimicrobial agents should have a broad spectrum of antimicrobial activity and be non-irritating to the eye. Some of the most common antimicrobial agents used in ophthalmic applications include benzalkonium chloride, chlorhexidine, polyquaternium-1, poly(hexamethylene biguanide) and alexidine.

Each antimicrobial compound has its own degree of efficacy against a specific collection of microorganisms. Because a single antimicrobial compound may not be efficacious against all microorganisms of interest in a safe and effective concentration range, it is sometimes beneficial to introduce another antimicrobial compound into the formulation. The difficulty, however, arises in the appropriate selection of the two or more antimicrobial compounds because of unknown chemical interactions that can exist between the two compounds or with other formulation components, e.g., a potential interaction with a surfactant.

U.S. Patent Publication No. 2004/0059006 describes a disinfectant composition comprising 1-(2-ethylhexyl)glycerol ether and one or more aromatic alcohols such as aryloxyalkanols, oligoalkanol aryl ethers or arylalkanols. The composition is said to be useful for disinfecting a variety of surfaces including the surface of contact lenses. There is no description, however, of preparing an ophthalmic composition containing 1-(2-ethylhexyl)glycerol ether and a cationic antimicrobial component.

Combinations of polyols and antimicrobial components are also known for use in ophthalmic applications. For example, U.S. Pat. Nos. 6,319,464 and 6,949,218 disclose that low molecular weight amino alcohols such as 2-amino-2-methyl-1-propanol (AMP), 2-dimethylaminomethyl-1-propanediol (DMAMP), 2-amino-2-ethyl-1,3-propanediol (AEPD), 2-amino-2-methyl-1,3-propanediol (AMPD), and 2-amino-1-butanol (AB) can enhance the activity of antimicrobial agents such as various biguanides including chlorhexidine, alexidine and polyhexamethylene biguanide (PHMB), especially in the presence borate or borate/polyol buffers.

Compositions containing 1-(2-ethylhexyl)glycerol ether and polyhexamethylene biguanide (PHMB) or chlorhexidine are also reported to be useful in compositions suitable for disinfecting skin and for impregnating medical articles such as catheters. See, U.S. Pat. No. 6,846,846 and U.S. Patent Publication No. 2004/0208908.

The development of disinfecting ophthalmic compositions that are simple to use, effective against a broad spectrum of microorganisms and are non-toxic and do not cause ocular irritation is of great interest.

### SUMMARY OF THE INVENTION

An aqueous ophthalmic composition comprising a branched glycerol compound selected from the group consisting of a branched, glycerol monoalkyl ether, a branched, glycerol monoalkyl amine, a branched, glycerol monoalkyl sulfide, or any mixture thereof, present in a total amount of from 0.05 ppm to 1,000 ppm; a cationic antimicrobial component, and having an osmolality in a range from 200 mOsmol/kg to 400 mOsmol/kg. The presence of the branched glycerol compound enhances the biocidal efficacy of the aqueous ophthalmic composition containing one or more cationic antimicrobial components, and thereby provides an opportunity to use less of the cationic antimicrobial component, which can cause ocular irritation, in the composition.

### DETAILED DESCRIPTION OF THE INVENTION

The invention is directed to an aqueous ophthalmic composition comprising a branched glycerol compound selected from the group consisting of a branched, glycerol monoalkyl ether, a branched, glycerol monoalkyl amine, a branched, glycerol monoalkyl sulfide, or any mixture thereof, present in a total amount of from 0.05 ppm to 1,000 ppm, and a cationic antimicrobial component. The ophthalmic composition will have an osmolality in a range from 200 mOsmol/kg to 400 mOsmol/kg. As used herein, the term “ophthalmic composition” denotes a composition intended for application in the eye or intended for treating a device to be placed in contact with the eye such as a contact lens.

In one embodiment, the ophthalmic composition comprises a branched, glycerol monoalkyl ether. In another embodiment, the ophthalmic composition comprises a branched, glycerol monoalkyl amine. In another embodiment, the ophthalmic composition comprises a branched, glycerol monoalkyl sulfide. In still another embodiment, the ophthalmic composition comprises any one mixture of a branched, glycerol monoalkyl ether, a branched, glycerol monoalkyl amine or a branched, glycerol monoalkyl sulfide.

In one embodiment, the branched, glycerol monoalkyl ether is 3-[(2-ethylhexyl)oxy]-1,2-propanediol (EHOPD). In another embodiment, the branched, glycerol monoalkyl amine is 3-[(2-ethylhexyl)amino]-1,2-propanediol (EHAPD). In another embodiment, the branched, glycerol monoalkyl sulfide is 3-[(2-ethylhexyl)thio]-1,2-propanediol (EHSPD). In still another embodiment, the ophthalmic composition comprises any one mixture of EHOPD, EHAPD and EHSPD. The chemical structures of EHOPD, EHAPD and EHSPD are provided below.

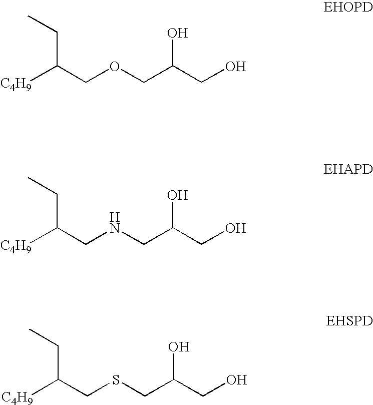

EHOPD is also referred to as octoxyglycerin and is sold under the tradename Sensiva® SC50 (Schtilke & Mayr). EHOPD is a branched, glycerol monoalkyl ether known to be gentle to the skin, and to exhibit antimicrobial activity against a variety of Gram-positive bacteria such as Micrococcus luteus, Corynebacterium aquaticum, Corynebacteriumflavescens, Corynebacterium callunae, and Corynebacterium nephredi. Accordingly, EHOPD is used in various skin deodorant preparations at concentrations between about 0.2 and 3 percent by weight. EHAPD can be prepared from 2-ethylhexylamine and 2,3-epoxy-1-propanediol using chemistry well known to those of ordinary skill in the art. EHSPD can be prepared from 2-ethylhexylthiol and 2,3-epoxy-1-propanediol using chemistry well known to those of ordinary skill in the art.

The cationic antimicrobial components include chemicals which derive their antimicrobial activity through a chemical or physiochemical interaction with microbes or microorganisms normally associated with contaminating a contact lens. Suitable antimicrobial components include, but are not limited to, quaternary ammonium salts used in ophthalmic applications such as α-[4-tris(2-hydroxyethyl)ammonium chloride-2-butenyllpoly[1-dimethylammonium chloride-2-butenyl]-ω-tris(2-hydroxyethyl) ammonium chloride (CAS#68518-54-7, available as Polyquaternium-1® from Onyx Corporation), myristamidopropyl dimethylamine (Aldox®), benzalkonium halides, and biguanides such as salts of alexidine, alexidine-free base, salts of chlorhexidine, hexamethylene biguanides and salts thereof and their polymers, antimicrobial polypeptides and mixtures thereof.

The term “cationic” when referring to an antimicrobial component refers to the predominant form of the antimicrobial component at neutral pH having a positive charge and a counteranion. An exemplary list of cationic disinfecting antimicrobial components include poly[dimethylimino-2-butene-1,4-diyl]chloride, α-[4-tris(2-hydroxyethyl) ammonium chloride-2-butenyl]poly[1-dimethylammonium chloride-2-butenyl]-ω-tris(2-hydroxyethyl)ammonium chloride, myristamidopropyl dimethylamine, poly(hexamethylene biguanide) (PHMB), PHMB-CG* and any mixture thereof.

PHMB is best described as a polymeric biguanide composition comprising at least six biguanide polymers each with a different combination of terminal guanidine, cyanoguanidino or amine terminal groups. Accordingly, a commercial sample of PHMB will likely comprise a mixture of various polymeric biguanides with the three mentioned terminal groups. The bigunaides differ with respect to which terminal groups are arranged on the polymer and what are the molar concentrations of each terminal group in the mixture. PHMB (Cosmocil® type PHMB) can contain from 20 mol % to 30 mol % terminal amine groups. The molar concentration of terminal guanidine groups and terminal cyanoguanidino groups range from 38 mol % to 49 mol % and 30 mol % to 32 mol %, respectively

A new synthetic route to polymeric biguanide compositions is described in copending U.S. provisional application Ser. No. 60/853,579 filed Oct. 23, 2006, and Ser. No. 60/895,770 filed Mar. 20, 2007. The new synthetic route provides a polymeric biguanide composition comprising less than 18 mol % of terminal amine groups as measured by ¹³C NMR. The polymeric biguanide composition also is characterized by a relative increase in the molar concentration of terminal guanidine groups or terminal cyanoguanidino groups. For example, in one embodiment, the biguanide composition comprises less than 18 mol % of terminal amine groups, and 55 mol % or greater of terminal guanidine groups. In another embodiment, the biguanide composition comprises less than 18 mol % of terminal amine groups, and 40 mol % or greater of terminal cyanoguanidino groups.

In this application we refer to this novel polymeric biguanide composition as PHMB-CG*. We also refer to polymeric biguanide compositions in the generic sense as “hexamethylene biguanides”, which one of ordinary skill in the art would recognize to include both PHMB as well as PHMB-CG*.

The cationic antimicrobial component is present in an amount from 0.01 ppm to 100 ppm, from 0.1 ppm to 50 ppm or from 0.1 ppm to 10 ppm. It is preferred, however, that the amount of antimicrobial component that is used is effective in disinfecting contact lenses contacted with the compositions, while at the same time promote lens patient comfort and acceptability.

In one embodiment, the primary antimicrobial component present in the lens care compositions is poly(hexamethylene biguanide) or PHMB-CG*, which is present from 0.01 ppm to 3 ppm. In another embodiment, the primary antimicrobial component present in the lens care compositions is α-[4-tris(2-hydroxyethyl)ammonium chloride-2-butenyl]poly[1-dimethylammonium chloride-2-butenyl]-ω-tris(2-hydroxyethyl) ammonium chloride, which is present from 1 ppm to 100 ppm.

Any one mixture of the two cationic antimicrobial components can also be present in the lens care compositions. For example, a particular lens care composition can include from 0.3 ppm to 0.8 ppm PHMB or PHMB-CG*, and 10 ppm to 60 ppm α-[4-tris(2-hydroxyethyl)ammonium chloride-2-butenyllpoly[1-dimethylammonium chloride-2-butenyl]-ω-tris(2-hydroxyethyl) ammonium chloride.

The lens care compositions can also include dexpanthenol, which is an alcohol of pantothenic acid, also called Provitamin B5, D-pantothenyl alcohol or D-panthenol. In some formulations of the lens care compositions, dexpanthenol can exhibit good cleansing action and can stabilize the lachrymal film at the eye surface when placing a contact lens on the eye. Dexpanthenol is preferably present in the contact lens care compositions in an amount from 0.2% to 10% (w/v), from 0.5% to 5% (w/v), or from 1% to 3% (w/v).

The lens care compositions can also include sorbitol, which is a hexavalent sugar alcohol. Typically, dexpanthenol is used in combination with sorbitol. In specific formulations the combination dexpanthenol and sorbitol can provide enhanced cleansing action and can also stabilize the lachrymal film following placement of the contact lens on the eye. These formulations can substantially improve patient comfort when wearing contact lenses. Sorbitol is present in the lens care compositions in an amount from 0.4% to 10% (w/v), from 0.8% to 6% (w/v) or from 1% to 3% (w/v).

The lens care compositions can also include one or more neutral or basic amino acids. The neutral amino acids include: the alkyl-group-containing amino acids such as alanine, isoleucine, valine, leucine and proline; hydroxyl-group-containing amino acids such as serine, threonine and 4-hydroxyproline; thio-group-containing amino acids such as cysteine, methionine and asparagine. Examples of the basic amino acid include lysine, histidine and arginine. The one or more neutral or basic amino acids are present in the compositions at a total concentration of from 0.1 % to 5% (w/v).

The lens care compositions can also include glycolic acid, aspartic acid or any mixture of the two at a total concentration of from 0.001 % to 4% (w/v) or from 0.01 % to 2.0% (w/v).

Further, the combined use of one or more amino acids and glycolic acid and/or aspartic acid can lead to a reduction in the change of the size of the contact lens due to swelling and shrinkage following placement of the lens on the eye. The stated combination provides a higher degree of compatibility with the contact lens compared to the absence of one of the two components in the composition. It is believed that one or more of the amino acids can cause the lens to swell, and that the glycolic acid and/or aspartic acid can cause the contact lens to shrink. If used in combination, however, a mutual counteraction of the two observed affects is believed to exist.

The lens care compositions can also include glycolic acid, aspartic acid or any mixture of the two, in combination with 2-amino-2-methyl-1,3-propanediol or a salt thereof. One observed advantage is that compositions that contain a mixture of two of the three, or all three, compounds minimizes the change of the lens size following placement of the contact lens in the eye. It is also believed that the stated combination of compounds minimizes the amount of uptake of the cationic antimicrobial component, particularly, α-[4-tris(2-hydroxyethyl)ammonium chloride-2-butenyllpoly[1-dimethylammonium chloride-2-butenyl]-ω-tris(2-hydroxyethyl)ammonium chloride, myristamidopropyl dimethylamine, benzalkonium halides, alexidine and salts thereof, salts of chlorhexidine, hexamethylene biguanides and salts thereof and their polymers such as poly(hexamethylene biguanide) or PHMB-CG*.

The 2-amino-2-methyl-1,3-propanediol (AMPD) or the salt thereof is added to the compositions in an amount to satisfy a predetermined molar ratio of glycolic acid, aspartic acid or any mixture of the two and AMPD. The molar ratio of the two components glycolic acid and/or aspartic acid to AMPD is 1:20 to 1.3:1. The glycolic acid, aspartic acid or any mixture of the two is present in the compositions at a concentration of 0.01% to 5% (w/v) or at a concentration of 0.05% to 1% (w/v).

If the components glycolic acid and/or aspartic acid, and AMPD, are present in the compositions in the absence of the other, one may observe a tendency to cause shrinkage or swelling of the lens. However, if these two components are combined together and used in the predetermined molar ratio, little, if any, change in the size of the lens is observed.

The amount of AMPD present in the compositions can be determined according to the amount of glycolic acid and/or aspartic acid in the composition. As stated, AMPD is present in an amount to provide a molar ratio of glycolic acid and/or aspartic acid to AMPD to be from 1:20to 1.3:1,from 1:15 to 1.2:1 or from 1:14 to 1:1. If the amount of AMPD exceeds 20 mols per 1 mol of glycolic acid and/or aspartic, adsorption of the cationic antimicrobial component on the contact lens will occur. If the amount of AMPD is less than 1 mol per 1.3 mols of glycolic acid and/or aspartic acid, a reduction in antimicrobial efficacy of the composition is observed.

As noted, the ophthalmic compositions are aqueous compositions and contain predominantly water. Accordingly, the aqueous ophthalmic compositions will generally comprise at least about 80 wt % of water, more preferably at least about 90 wt % of water.

The aqueous ophthalmic compositions will also generally contain at least one component, and preferably a number of other components, in addition to the antimicrobial/preservative system. The additional component(s) will comprise one or more ophthalmic composition adjuvants. Such ophthalmic composition adjuvants can be selected from the group consisting of surfactants, buffers, viscosity control/wetting agents, sequestering agents, tonicity agents, ancillary antimicrobial agents, and combinations of such adjuvants.

One of the most common ophthalmic composition adjuvants comprises a suitable surfactant having known advantages in terms of cleaning efficacy and comfort. Surfactants may be present in the subject ophthalmic compositions in a total amount of from about 0.01 wt % to about 5.0 wt %, but more preferably from about 0.1 wt % to about 0.5 wt %. Suitable surfactants for use in the present compositions should be soluble in the ophthalmic compositions, should not become turbid, and should be non-irritating to eye tissues. Suitable surfactants include, but are not limited to polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO materials are commercially available under the trade names Pluronics™, R-Pluronics™, Tetronics™ and R-Tetronics™ (BASE Wyandotte Corp., Wyandotte, Mich.) and are further described in U.S. Pat. No. 4,820,352 incorporated herein by reference. The classes of nonionic polyalkoxylated copolymers known by the Poloxamer and Poloxamine tradenames can also be used.

Another common ophthalmic composition adjuvant comprises a suitable buffer or buffer system to maintain the compositions herein within a suitable pH range. Suitable buffers include, for example, but are not limited to amino alcohol buffers, phosphate buffers, borate buffers, tris(hydroxymethyl)aminomethane (Tris) buffers, bis(2-hydroxyethyl)-imino-tris(hydroxymethyl)methane (bis-Tris) buffers, citrate buffers, sodium bicarbonate, and combinations thereof. A suitable buffering system, for example, may include at least one phosphate buffer and at least one borate buffer. Buffering agents and systems of the foregoing types are described in greater detail in PCT Patent Application Nos. WO 04/093545 and WO 05/053759, both of which are incorporated herein by reference. Buffers can generally comprise from about 0.01 wt % to about 2.0 wt % of the compositions herein. The pH of ophthalmic compositions of the present invention is preferably maintained within the range of 5.0 to 8.0, more preferably about 6.0 to 8.0, most preferably about 6.5 to 7.8.

Yet another suitable ophthalmic composition component comprises one or more viscosity control/wetting agents. Because of the demulcent effect of viscosity control and wetting agents, these materials have a tendency to enhance a contact lens wearer's comfort by means of a film on the lens surface cushioning impact against the eye. Suitable viscosity control/wetting agents include, for example, but are not limited to cellulose polymers like hydroxyethylcellulose or hydroxypropylcellulose, polyquatemium-10, and carboxymethylcellulose; povidone; poly(vinyl alcohol), poly(ethylene oxide) and poly(N,N-dimethylacrylamide) and the like. Viscosity control and wetting agents of the foregoing types are also described in greater detail in PCT Patent Application Nos. WO 04/093545 and WO 05/053759, both of which are again incorporated herein by reference. Viscosity control/wetting agents may be employed in the ophthalmic compositions in amounts ranging from about 0.001 wt % to about 1.0 wt % or less.

Yet another suitable ophthalmic composition component comprises one or more sequestering agents. Sequestering agents serve to bind metal ions which, in the case of ophthalmic solutions, might otherwise react with protein deposits and collect on contact lenses. Suitable sequestering agents include, for example, but are not limited to ethylenediaminetetraacetic acid (EDTA) and its salts. If used, sequestering agents may be added to the ophthalmic compositions herein in amounts ranging from about 0.01 wt % to about 5.0 wt %.

The lens care compositions can also include a phosphonic acid, or its physiologically compatible salt, that is represented by the following formula:

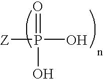

wherein Z is a connecting radical equal, n is an integer from 1 to 4, or 1, 2 or 3, and preferably containing 1 to 12 carbon atoms, more preferably 3 to 10 carbon atoms. The Z radical comprises substituted or unsubstituted saturated hydrocarbon radicals or amine-containing radicals, which amine-containing radicals are saturated hydrocarbon radicals in which the carbon atoms are interrupted with at least one nitrogen atom such as 1, 2 or 3 nitrogen atoms that forms a secondary or tertiary amine.

Accordingly, suitable Z radicals include substituted or unsubstituted alkylidene, substituted or unsubstituted alkylene, amino tri(alkylene) having at least n+1 carbon atoms, amino di(alkylene) having at least n+1 carbon atoms, alkylenediaminetetra(alkylene) or a dialkylenetriamine penta(alkylene) radical. In each case, the alkylene group in parenthesis is connected to a phosphonic acid group. Preferably, all alkylene groups independently have 1 to 4 carbon atoms.

Exemplary compounds in which the Z group is an amino tri(alkylene) radical includes amino tri(ethylidene phosphonic acid), amino tri(isopropylidene phosphonic acid), amino di(methylene phosphonic acid) mono(isopropylidene phosphonic acid), and amino mono(methylene phosphonic acid) di(ethylidene phosphonic acid). Exemplary compounds in which the Z group is a substituted or unsubstituted alkylidene radical includes methylene diphosphonic acid, ethylidine diphosphonic acid, 1-hydroxy propylidene diphosphonic acid. Exemplary compounds in which the Z group is an alkylenediaminetetra(alkylene) or a dialkylenetriamine penta(alkylene) radical include hexamethylenediaminetetra(methylene phosphonic acid) and diethylenetriaminepenta(methylenephosphonic acid).

In one embodiment, the phosphonic acid, or its physiologically compatible salt, is represented by the following formula:

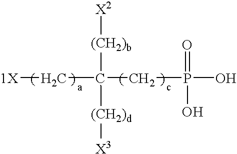

wherein each of a, b, c, and d are independently selected from integers from 0 to 4, preferably 0 or 1; X¹ is a phosphonic acid group (i.e., P(OH)₂O), hydroxy, amine or hydrogen; and X² and X³ are independently selected from the group consisting of halogen, hydroxy, amine, carboxy, alkylcarbonyl, alkoxycarbonyl, or substituted or unsubstituted phenyl, and methyl. Exemplary substituents on the phenyl are halogen, hydroxy, amine, carboxy and/or alkyl groups. A particularly preferred species is that wherein a, b, c, and d in are zero, specifically the tetrasodium salt of 1-hydroxyethylidene-1,1-diphosphonic acid, also referred to as tetrasodium etidronate, commercially available from Monsanto Company as DeQuest® 2016 diphosphonic acid sodium salt or phosphonate.

Yet another suitable ophthalmic composition component comprises one or more tonicity agents. Tonicity agents (also called osmolality-adjusting agents) serve to have the compositions herein approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride; monosaccharides such as dextrose, mannose, sorbitol and mannitol; low molecular weight polyols such as glycerin and propylene glycol; and calcium and magnesium chloride. These tonicity agents are typically used individually in the ophthalmic compositions herein in amounts ranging from about 0.01 wt % to about 2.5 percent wt %.

The ophthalmic compositions herein can be formulated by combining the essential and preferred components in the requisite amounts in any suitable order and in any conventional manner for formulations of this type. Generally such compositions can be prepared by adding the active or functional components to deionized water under conditions which dissolve or disperse those components in the aqueous compositions.

The compositions are used as a disinfecting solution, a preservative solution or packaging solution for contact lenses including (1) hard lenses formed from materials prepared by polymerization of acrylic esters such as polymethyl methacrylate (PMMA), (2) rigid gas permeable (RGP) lenses formed from silicone acrylates and fluorosilicone methacrylates, (3) soft, hydrogel lenses, and (4) non-hydrogel elastomer lenses.

As an example, soft hydrogel contact lenses are made of a hydrogel polymeric material, a hydrogel being defined as a crosslinked polymeric system containing water in an equilibrium state. In general, hydrogels exhibit excellent biocompatibility properties, i.e., the property of being biologically or biochemically compatible by not producing a toxic, injurious or immunological response in a living tissue. Representative conventional hydrogel contact lens materials are made by polymerizing a monomer mixture comprising at least one hydrophilic monomer, such as (meth)acrylic acid, 2-hydroxyethyl methacrylate (HEMA), glyceryl methacrylate, N,N-dimethacrylamide, and N-vinylpyrrolidone (NVP). In the case of silicone hydrogels, the monomer mixture from which the copolymer is prepared further includes a siloxy-containing monomer, in addition to the hydrophilic monomer. Generally, the monomer mixture will include a crosslinking monomer, i.e., a monomer having at least two polymerizable radicals, such as ethylene glycol dimethacrylate, tetraethylene glycol dimethacrylate, and methacryloxyethyl vinylcarbonate. Alternatively, either the siloxy-containing monomer or the hydrophilic monomer may function as a crosslinking agent.

The invention relates is also directed to a method of treating a patient with dry eye using the aqueous ophthalmic compositions described herein. The method includes administering the ophthalmic composition to the eye, eye lid or to the skin surrounding the eye. The compositions are thus useful for relieving eye irritation or dryness and providing lubrication for the eyes, irrespective of whether contact lenses are present in the eyes of the patient.

The ophthalmic compositions can be formulated to function as artificial tears and can be used, as needed, for the temporary relief of eye irritation or discomfort. For example, many people suffer from temporary or chronic eye conditions in which the eye's tear system fails to provide adequate tear volume or tear film stability necessary to remove irritating environmental contaminants such as dust, pollen, or the like. In persons suffering from chronic dry eye, the film on the eye tends to becomes discontinuous. The ophthalmic compositions can be used to treat the above conditions.

The invention is also directed to a method for preserving, disinfecting or cleaning contact lenses. In general, such a method comprises contacting the lenses with an ophthalmic composition. Although such contacting may be accomplished by simply soaking a lens in the ophthalmic composition, greater preserving, disinfecting or cleaning may possibly be achieved if a few drops of the composition are initially placed on each side of the lens, and the lens is rubbed for a period of time, for example, approximately 20 seconds. The lens can then be subsequently immersed within several milliliters of the subject composition. Preferably, the lens is permitted to soak in the composition for at least four hours. Furthermore, the lens is preferably rinsed with fresh composition after any rubbing step and again after being immersed within the composition. The lenses can then be removed from the composition, rinsed with the same or a different liquid, for example, a preserved isotonic saline solution and placed on the eye.

The biocidal efficacy of ophthalmic compositions is illustrated by the following examples.

### Preservative/Disinfectant System Efficacy Testing

The ophthalmic compositions are evaluated in accordance with specific procedures outlined by the US Food & Drug Administration and the International Organization for Standardization for the testing of microbiocidal efficacy. Such procedures are described in documents entitled Premarket Notifiction (510(k)) Guidance Document for Contact Lens Care Products (May, 1997) and ISO/DIS 14729 Ophthalmic Optics-Contact Lens Care Products Microbiological. The microorganisms challenged in this procedure include: Pseudomonas aeruginosa (ATCC 9027), Staphylococcus aureus (ATCC 6538), Serratia marcescens (ATCC 13880), Candida albicans (ATCC 10231) and Fusarium solani (ATCC 36031).

### EXAMPLE 1

The log reduction of microorganisms with 10% organic soil for 3-[(2-ethylhexyl)oxy]-1,2-propanediol (EHOPD) in borate buffered saline is shown in Table 1

TABLE 1
Biocidal data for EHOPD in Borate Buffered Saline

| Conc. | Time | S. | P. | S. | C. | F. |
|---|---|---|---|---|---|---|
| (wt %) | (hr) | aureus | aeruginosa | marcescens | albicans | solani |
| 0.0001 | 1 | 1.4 | 1.5 | 1.7 | 2.3 | 1.3 |
| | 4 | 1.4 | 1.5 | 1.7 | 2.3 | 2.1 |
| 0.001 | 1 | 1.4 | 1.5 | 1.7 | 2.3 | 1.9 |
| | 4 | 1.4 | 1.5 | 1.7 | 2.3 | 1.9 |
| 0.01 | 1 | 1.4 | 2.6 | 1.7 | 2.3 | 2.4 |
| | 4 | 1.4 | >5.0 | 1.7 | 2.3 | >5.8 |
| 0.1 | 1 | 1.7 | >5.0 | 4.8 | 5.4 | >5.8 |
| | 4 | 4.0 | >5.0 | >5.2 | >5.7 | >5.8 |
| 0.5 | 1 | 1.8 | >5.0 | 4.9 | 5.1 | >5.8 |
| | 4 | 4.3 | >5.0 | >5.2 | >5.7 | >5.8 |

### EXAMPLE 2

The log reduction of microorganisms with 10% Organic Soil for EHOPD in phosphate buffered saline is shown in Table 2.

TABLE 2
Biocidal Data for EHOPD in Phosphate Buffered Saline

| Conc. | Time | S. | P. | S. | C. | F. |
|---|---|---|---|---|---|---|
| (wt %) | (hr) | aureus | aeruginosa | marcescens | albicans | solani |
| 0.0001 | 1 | 1.4 | 1.5 | 1.7 | 2.3 | 2.3 |
| | 4 | 1.4 | 1.5 | 1.7 | 2.3 | 2.3 |
| 0.001 | 1 | 1.4 | 1.5 | 1.7 | 2.3 | 1.8 |
| | 4 | 1.4 | 1.5 | 3.2 | 2.3 | 2.9 |
| 0.01 | 1 | 1.4 | 1.5 | >5.2 | 2.3 | 3.6 |
| | 4 | 1.4 | 1.5 | >5.2 | 2.3 | >5.8 |
| 0.1 | 1 | 1.4 | 1.5 | >5.2 | 3.8 | >5.8 |
| | 4 | 3.0 | 3.7 | >5.2 | >5.7 | >5.8 |
| 0.5 | 1 | 1.4 | 1.5 | >5.2 | 3.8 | >5.8 |
| | 4 | 3.1 | 4.1 | >5.2 | >5.7 | >5.8 |

### EXAMPLE 3

The biocidal efficacy of EHOPD is also evaluated for multi-purpose lens care formulation detailed in Table 3A. The log reduction values of microorganisms with 10% organic soil for the multi-purpose lens care formulations of Table 3A with varying concentrations of EHOPD are detailed in Table 3B.

TABLE 3A

| Compound | (wt %) |
|---|---|
| Sodium Phosphate (monobasic) | 0.23 |
| Sodium Phosphate (dibasic) | 0.77 |
| Sodium Chloride | 0.19 |
| (30%) Hydroxyalkylphosphonate | 0.10 |
| Poloxamer 407 | 2.0 |
| Poloxamine 1107 | 1.0 |
| Polyquaterium-10 | 0.02 |
| EHOPD | 0.01 to 1.0 |
| Water | Q.S. to 100% |

The biocidal data from Examples 1, 2 and 3 indicate that EHOPD exhibits significant activity against all of the microorganisms in the study, and thus is useful as an antimicrobial component for preservation or disinfection efficacy in ophthalmic compositions.

### EXAMPLE 4

The log reduction of microorganisms with 10% organic soil for lens care formulations comprising EHOPD plus alexidine in phosphate buffered saline is detailed in Table 4.

TABLE 3B
Biocidal Data for lens care formulations of Example 3.

| Conc. | Time | S. | P. | S. | C. | F. |
|---|---|---|---|---|---|---|
| (wt %) | (hr) | aureus | aeruginosa | marcescens | albicans | solani |
| 0.01 | 1 | 1.4 | 1.9 | 2.0 | 2.0 | 0.3 |
| | 4 | 1.4 | 1.9 | 2.0 | 2.0 | 0.5 |
| 0.1 | 1 | 1.4 | >5.4 | 2.0 | 2.0 | 0.3 |
| | 4 | 1.4 | >5.4 | 2.0 | 2.0 | 0.5 |
| 0.15 | 1 | 1.4 | >5.4 | 2.0 | 2.0 | 0.5 |
| | 4 | 1.4 | >5.4 | 4.7 | 2.0 | >4.7 |
| 0.25 | 1 | 1.4 | >5.4 | >5.4 | 2.0 | 0.8 |
| | 4 | 1.4 | >5.4 | >5.4 | 2.0 | >4.7 |
| 0.50 | 1 | 1.4 | >5.4 | >5.4 | 2.0 | >4.7 |
| | 4 | 1.8 | >5.4 | >5.4 | 3.1 | >4.7 |
| 0.75 | 1 | 1.6 | >5.4 | >5.4 | 4.2 | >4.7 |
| | 4 | 3.1 | >5.4 | >5.4 | >5.5 | >4.7 |
| 1.0 | 1 | 2.2 | >5.4 | >5.4 | >5.5 | >4.7 |
| | 4 | 3.7 | >5.4 | >5.4 | >5.5 | >4.7 |

TABLE 4
Biocidal data for EHOPD with alexidine in phosphate buffered saline

| Conc. | Time | S. | | S. | | F. |
|---|---|---|---|---|---|---|
| (wt %) | (hr) | aureus | P. aeruginosa | marcescens | C. albicans | solani |
| 2.0 ppm Alex. | 1 | 1.4 | 1.6 | 2.0 | 3.2 | 2.3 |
| | 4 | 1.4 | 2.3 | 3.5 | 3.8 | 2.3 |
| 0.20% EHOPD | 1 | 1.2 | 1.5 | 3.0 | >5.0 | 2.5 |
| | 4 | 1.3 | 1.5 | 3.9 | >5.0 | 5.0 |
| 2.0 ppm Alex. | 1 | 1.7 | 1.6 | 2.6 | >5.0 | 3.4 |
| 0.10% EHOPD | 4 | 1.7 | 3.1 | 4.0 | >5.0 | >5.4 |
| 2.0 ppm Alex. | 1 | 2.1 | 1.8 | 2.0 | 5.2 | 4.0 |
| 0.20% EHOPD | 4 | 4.1 | 3.8 | 4.4 | >5.2 | >5.4 |
| 2.0 ppm Alex. | 1 | 2.0 | 3.5 | 2.3 | >5.2 | 4.4 |
| 0.30% EHOPD | 4 | 4.5 | >5.2 | 4.8 | >5.2 | >5.4 |

The Table 4 data indicate that the biocidal efficacy of the combination of disinfectants is more effective against all five challenged microorganism than is either EHOPD or the alexidine alone.

### EXAMPLE 5

The biocidal efficacy of EHOPD plus alexidine is evaluated in multi-purpose lens care solutions having the formulation shown in Table 5A.

TABLE 5A

| Compound | (% w/w) |
|---|---|
| Sodium Phosphate (monobasic) | 0.23 |
| Sodium Phosphate (dibasic) | 0.77 |
| Sodium Chloride | 0.19 |
| HAP (30%) Hydroxyalkylphosphonate | 0.10 |
| Poloxamer 407 | 2.0 |
| Poloxamine 1107 | 1.0 |
| Polyquaterium-10 | 0.02 |
| 3-[(2-ethylhexyl)oxy]-1,2-propanediol | 0.0–0.3 |
| Alexidine | 0–3.0 ppm |
| Water | Q.S. to 100% |

The log reduction of microorganisms with 10% organic soil is evaluated for the formulation of Example 5.

TABLE 5B
Biocidal data for lens care formulations of Example 5

| | Time | | | | | |
|---|---|---|---|---|---|---|
| Disinfectant | (hr) | S. aureus | P. aeruginosa | S. marcescens | C. albicans | F. solani |
| 3.0 ppm alex. | 1 | 3.3 | 3.5 | 4.1 | 2.0 | 3.1 |
| | 4 | 3.8 | 3.9 | >5.2 | 3.0 | 4.2 |
| 0.20% EHOPD | 1 | 1.2 | 1.4 | 2.3 | 2.1 | 3.1 |
| | 4 | 1.2 | 1.4 | 3.3 | 2.8 | 5.0 |
| 3.0 ppm alex. | 1 | 2.8 | 3.5 | 3.9 | 2.3 | >5.0 |
| 0.10% EHOPD | 4 | 3.6 | 4.0 | >5.2 | 3.5 | >5.0 |
| 3.0 ppm alex. | 1 | 3.4 | 4.2 | 4.8 | 2.7 | >5.0 |
| 0.20% EHOPD | 4 | 4.6 | >5.4 | >5.2 | 3.2 | >5.0 |
| 3.0 ppm Alex. | 1 | 4.2 | >5.4 | >5.2 | 3.4 | >5.0 |
| 0.30% EHOPD | 4 | 5.2 | >5.4 | >5.2 | 4.5 | >5.0 |

The biocidal data of Table 5B indicates that the EHOPD/alexidine lens care formulations exhibit greater biocidal activity against all of the microorganisms investigated in comparison to EHOPD or alexidine alone. These results are similar to those which are obtained for the combination of antimicrobial agents in phosphate buffered saline as shown in Table 4.

### EXAMPLE 6

The cyto-toxicity profile of Example 5 is evaluated using a model in vitro sodium fluorescein permeability assay, which is used in the art to evaluate the affect, if any, on the epithelium. The procedures used in this assay are those described in Tchao et al, CLAO Journal 2002, 28(3), 151-156.

This cytotoxicity assay involves exposing a monolayer of Madin-Darby canine kidney cells to various test solutions for 30 minutes. The monolayer of cells that are exposed to the test solutions are then treated with sodium fluorescein. The sodium fluorescein that penetrates the tight junctions of the cells is detected using fluorescence spectroscopy. The fluorescence analysis is repeated after 24 hours of incubation to evaluate the recovery of the epithelial monolayer. Table 6 outlines cytotoxicity profiles for various test solutions including the formulation of Example 5.

TABLE 6

| | Fluorescence | Fluorescence |
|---|---|---|
| | Intensity (a.u.) | Intensity (a.u.) |
| Test Solution | 30 min | 24 hr |
| Hank's Balanced Salt Solution¹ | 62 ± 5 | 39 ± 6 |
| 0.05% Sodium dodecyl sulfate | 1429 ± 11 | 1970 ± 500 |
| Opti-Free ® Express ®² | 307 ± 38 | 57 ± 10 |
| Table 5A solution with 3.0 ppm | 110 ± 45 | 46 ± 5 |
| alexidine | | |
| Table 5A solution with 0.20% | 116 ± 6 | 87 ± 8 |
| EHOPD | | |
| Table 5A solution with 3.0 ppm | 97 ± 10 | 75 ± 8 |
| alexidine + 0.10% EHOPD | | |
| Table 5A solution with 3.0 ppm | 122 ± 10 | 94 ± 11 |
| alexidine + 0.20% EHOPD | | |
| Table 5A solution with 3.0 ppm | 285 ± 11 | 104 ± 6 |
| alexidine + 0.30% EHOPD | | |

¹Mammalian tissue culture medium available from Biosure ®
²Opti-Free ® Express ® is a registered trademark of Alcon Laboratories, Inc.

The Table 6 sodium fluorescein permeability assay data indicate that within a certain concentration range, the EHOPD-based dual disinfection systems of the present invention impart low cytotoxcity to the epithelium in comparison to sodium dodecyl sulfate solution and Opti-Free® Express®. The Table 6 assay results further indicate that these solutions impart low toxicity to the epithelial monolayer.

### EXAMPLE 7

The log reduction of microorganisms in a composition comprising 3-[(2-ethylhexyl)amino]-1,2-propanediol (EHAPD) plus alexidine in phosphate buffered saline is shown in Table 7. The Table 7 data indicate that the biocidal efficacy of the compositions is more effective against all five challenged microorganism than either EHAPD or alexidine alone.

TABLE 7
Biocidal data for EHAPD plus alexidine in phosphate buffered saline

| | Time | S. | P. | | | F. |
|---|---|---|---|---|---|---|
| Conc. % w/w | (hr) | aureus | aeruginosa | S. marcescens | C. albicans | solani |
| 2.0 ppm Alex. | 1 | 1.4 | 1.6 | 2.0 | 3.2 | 2.3 |
| | 4 | 1.4 | 2.3 | 3.5 | 3.8 | 2.3 |
| 0.20% EHAPD | 1 | 1.4 | 1.4 | 3.3 | >5.0 | 2.3 |
| | 4 | 1.4 | 1.4 | 3.8 | >5.0 | 5.2 |
| 2.0 ppm Alex. | 1 | 1.4 | 1.6 | 2.6 | >5.0 | 3.5 |
| 0.10% EHAPD | 4 | 1.4 | 3.1 | 3.5 | >5.0 | >5.4 |
| 2.0 ppm Alex. | 1 | 1.7 | 1.7 | 2.2 | 4.8 | 4.6 |
| 0.20% EHAPD | 4 | 4.0 | 3.5 | 4.2 | >5.2 | >5.4 |
| 2.0 ppm Alex. | 1 | 1.8 | 3.3 | 2.5 | >5.2 | 4.8 |
| 0.30% EHAPD | 4 | 4.3 | >5.2 | 4.6 | >5.2 | >5.4 |

### EXAMPLE 8

The biocidal efficacy of EHAPD plus alexidine is also evaluated for multi-purpose lens care solutions having the formulation shown in Table 8A.

TABLE 8A

| Compound | (% w/w) |
|---|---|
| Sodium Phosphate (monobasic) | 0.23 |
| Sodium Phosphate (dibasic) | 0.77 |
| Sodium Chloride | 0.19 |
| HAP (30%) Hydroxyalkylphosphonate | 0.10 |
| Poloxamer 407 | 2.0 |
| Poloxamine 1107 | 1.0 |
| Polyquaterium-10 | 0.02 |
| 3-[(2-ethylhexyl)amino]-1,2-propanediol | 0.0–0.30 |
| Alexidine | 3.0 ppm |
| Water | Q.S. to 100% |

The log reduction of microorganisms for the lens care formulations of Table 8A multi-purpose lens care formulation with varying concentrations of EHAPD and alexidine are shown in Table 8B. The biocidal data of Table 8B suggest that the compositions comprising EHAPD and alexidine provide superior biocidal activity against all of the microorganisms investigated in comparison to EHAPD and alexidine alone. These results are similar to those obtained for the compositions in phosphate buffered saline as shown in Table 7.

TABLE 8B
Biocidal data for EHAPD plus alexidine

| | Time | S. | | S. | | F. |
|---|---|---|---|---|---|---|
| Disinfectant | (hr) | aureus | P. aeruginosa | marcescens | C. albicans | solani |
| 3.0 ppm Alex. | 1 | 3.3 | 3.5 | 4.1 | 2.0 | 3.1 |
| | 4 | 3.8 | 3.9 | >5.2 | 3.0 | 4.2 |
| 0.20% EHAPD | 1 | 1.2 | 1.2 | 2.7 | 2.4 | 2.4 |
| | 4 | 1.2 | 1.3 | 3.2 | 2.6 | 4.8 |
| 3.0 ppm Alex. | 1 | 3.1 | 3.6 | 3.3 | 2.1 | >5.0 |
| 0.10% EHAPD | 4 | 3.8 | 4.1 | 3.8 | 3.0 | >5.0 |
| 3.0 ppm Alex. | 1 | 3.2 | 4.5 | 4.5 | 2.8 | >5.0 |
| 0.20% EHAPD | 4 | 4.4 | >5.4 | >5.2 | 3.4 | >5.0 |
| 3.0 ppm Alex. | 1 | 3.8 | >5.4 | >5.2 | 3.5 | >5.0 |
| 0.30% EHAPD | 4 | 4.8 | >5.4 | >5.2 | 4.0 | >5.0 |

### EXAMPLE 9

The cytotoxicity profile of Example 8 is evaluated using the same in vitro sodium fluorescin permeability assay described in Example 6. Table 9 outlines cytotoxicity profiles for various test solutions including the multi-purpose solutions of Table 8B. The Table 9 sodium fluorescence permeability assay data indicate that within a certain concentration range, the EHAPD plus alexidine compositions impart low cyto-toxcity to the epithelium in comparison to sodium dodecyl sulfate solution and Opti-Free® Express®. The Table 9 data further indicates that the compositions impart low toxicity to the epithelial monolayer.

TABLE 9
In Vitro Sodium Fluorescein Permeability Assay S

| | Fluorescence | Fluorescence |
|---|---|---|
| | Intensity (a.u.) | Intensity (a.u.) |
| Sample | 30 min | 24 hr |
| Hank's Balanced Salt Solution¹ | 57 ± 5 | 20 ± 6 |
| 0.05% Sodium dodecyl sulfate | 1430 ± 11 | 1970 ± 200 |
| Opti-Free ® Express ®² | 147 ± 21 | 91 ± 8 |
| Table 8A solution with 3.0 ppm | 54 ± 1 | 39 ± 8 |
| alexidine | | |
| Table 8A solution with 0.20% | 110 ± 16 | 85 ± 4 |
| EHAPD | | |
| Table 8A solution with 3.0 ppm | 105 ± 9 | 81 ± 6 |
| alexidine + 0.10% EHAPD | | |
| Table 8A solution with 3.0 ppm | 134 ± 22 | 97 ± 10 |
| alexidine + 0.20% EHAPD | | |
| Table 8A solution with 3.0 ppm | 138 ± 10 | 100 ± 6 |
| alexidine + 0.30% EHAPD | | |

¹Mammalian tissue culture medium available from Biosure ®
²Opti-Free ® Express ® is a registered trademark of Alcon Laboratories, Inc.

### EXAMPLE 10

PHMB (Polyaminopropyl biguanide hydrochloride, 2 g, 0.0011 mole,) and HMBDA (1,6-bis(cyanoguanadino)hexane, 0.3 g, 0.0012 mole were mixed and ground together, then placed in a 100 mL round bottom flask. Concentrated hydrochloric acid (100 μL) was then added to the PHMB/HMBDA. The mixture was slowly heated to 100° C. until all the liquid was driven off. The heat was then increased to 150° C. to 160° C. and held for 4 hours. The reaction mixture was cooled to room temperature providing 1.32 g of the crystalline material PHMB-CG*.

What is claimed is:

1. An aqueous ophthalmic composition comprising a branched glycerol compound selected from the group consisting of a branched, glycerol monoalkyl ether, a branched, glycerol monoalkyl amine, a branched, glycerol monoalkyl sulfide, or any mixture thereof, present in a total amount of from 0.05 ppm to 1,000 ppm; and
a cationic antimicrobial component, wherein the ophthalmic composition has an osmolality in a range from 200 mOsmol/kg to 400 mOsmol/kg.

2. The composition of claim 1 wherein the branched, glycerol monoalkyl compound is selected from the group consisting of 3-[(2-ethylhexyl)oxyl-1,2-propanediol, 3-[(2-ethylhexyl)amino]-1,2-propanediol, 3-[(2-ethylhexyl)thio]-1,2-propanediol or any mixture thereof.

3. The composition of claim 1 wherein the cationic antimicrobial component is selected from the group consisting of poly[dimethylimino-2-butene-1,4-diyl]chloride, α-[4-tris(2-hydroxyethyl)ammonium chloride-2-butenyl]poly[1-dimethylammonium chloride-2-butenyl]-ω-tris(2-hydroxyethyl)ammonium chloride, myristamidopropyl dimethylamine, benzalkonium halides, alexidine and salts thereof, hexamethylene biguanides and salts thereof and their polymers, and mixtures thereof.

4. The composition of claim 1 wherein the cationic antimicrobial component is selected from the group consisting of poly(hexamethylene biguanide) or PHMB-CG*, which is present from 0.01 ppm to 3 ppm, α-[4-tris(2-hydroxyethyl)ammonium chloride-2-butenyllpoly[1-dimethylammonium chloride-2-butenyl]ω)-tris(2-hydroxyethyl) ammonium chloride, which is present from 1 ppm to 100 ppm, and any mixture thereof.

5. The composition of claim 1 wherein the cationic antimicrobial component is poly(hexamethylene biguanide) or PHMB-CG*.

6. The composition of claim 1 further comprising dexpanthenol, sorbitol or any mixture thereof.

7. The composition of claim 1 further comprising 2-amino-2-methyl-1,3-propanediol, and glycolic acid, aspartic acid or a mixture thereof, wherein a molar ratio of the total glycolic acid, aspartic acid or mixture thereof to AMPD is from 1:20 to 1.3:1.

8. The use of the ophthalmic composition of claim 1 in an eye care or a contact lens care product selected from the group consisting of eye drops, contact lens preservative solution, contact lens packaging solution, and contact lens multi-purpose solution.

9. A method of enhancing the biocidal efficacy of an aqueous ophthalmic composition containing one or more cationic antimicrobial components, the method comprising adding a branched glycerol compound selected from the group consisting of a branched, glycerol monoalkyl ether, a branched, glycerol monoalkyl amine, a branched, glycerol monoalkyl sulfide, or any mixture thereof, to the composition in a total amount of from 0.05 ppm to 1,000 ppm, the composition having an osmolality in a range from 200 mOsmol/kg to 400 mOsmol/kg.

10. The method of claim 9 wherein the one or more cationic antimicrobial components are selected from the group consisting of poly[dimethylimino-2-butene-1,4-diyl]chloride, α-[4-tris(2-hydroxyethyl)ammonium chloride-2-butenyl]poly[1-dimethylammonium chloride-2-butenyl]-ω-tris(2-hydroxyethyl) ammonium chloride, myristamidopropyl dimethylamine, benzalkonium halides, alexidine and salts thereof, hexamethylene biguanides and salts thereof and their polymers.

11. The method of claim 9 wherein the cationic antimicrobial component is selected from the group consisting of poly(hexamethylene biguanide) or PHMB-CG*, which is present from 0.01 ppm to 3 ppm, α-[4-tris(2-hydroxyethyl)ammonium chloride-2-butenyl]poly[1-dimethylammonium chloride-2-butenyl]-ω-tris(2-hydroxyethyl) ammonium chloride, which is present from 1 ppm to 100 ppm, and any mixture thereof.

12. The method of claim 9 wherein the cationic antimicrobial component is poly(hexamethylene biguanide) or PHMB-CG*.

13. The method of claim 9 further comprising adding dexpanthenol, sorbitol or any mixture thereof to the ophthalmic composition.

14. The method of claim 9 further comprising adding 2-amino-2-methyl-1,3-propanediol, and glycolic acid, aspartic acid or a mixture thereof to the ophthalmic composition, wherein a molar ratio of the total glycolic acid, aspartic acid or mixture thereof to AMPD is from 1:20 to 1.3:1.

15. An aqueous ophthalmic composition comprising:
a branched glycerol compound selected from the group consisting of a branched, glycerol monoalkyl ether, a branched, glycerol monoalkyl amine, a branched, glycerol monoalkyl sulfide, or any mixture thereof, present in a total amount of from 0.05 ppm to 1,000 ppm;
a cationic antimicrobial component selected from the group consisting of poly(hexamethylene biguanide) or PHMB-CG*, which is present from 0.01 ppm to 3 ppm, α-[4-tris(2-hydroxyethyl)ammonium chloride-2-butenyllpoly[1-dimethyl ammonium chloride-2-butenyl]-ω-tris(2-hydroxyethyl)ammonium chloride, which is present from 1 ppm to 100 ppm, and any mixture thereof, wherein the ophthalmic composition has an osmolality in a range from 200 mOsmol/kg to 400 mOsmol/kg.

16. The composition of claim 15 further comprising dexpanthenol, sorbitol or any mixture thereof.

17. The composition of claim 15 further comprising 2-amino-2-methyl-1,3-propanediol, and glycolic acid, aspartic acid or a mixture thereof, wherein a molar ratio of the total glycolic acid, aspartic acid or mixture thereof to AMPD is from 1:20 to 1.3:1.
